(19) United States
(12) Patent Application Publication
(10) Pub. No.: US 2022/0062517 A1
(43) Pub. Date: Mar. 3, 2022

(54) METHOD OF PRIORITIZING COMMUNICATION CONNECTIONS FOR A FULLY IMPLANTED LVAD SYSTEM

(71) Applicant: Medtronic, Inc., Minneapolis, MN (US)

(72) Inventors: Eric A. Schilling, Ham Lake, MN (US); Bo Zhang, Blaine, MN (US); Thomas J. August, Minneapolis, MN (US); Joel B. Artmann, Elk River, MN (US); Christopher T. House, Pine Island, MN (US)

(21) Appl. No.: 17/008,824

(22) Filed: Sep. 1, 2020

(51) Int. Cl. A61M 1/12; A61M 1/10; H04W 76/10; H04L 12/18; H04W 76/30; H02J 50/80; H02J 50/10

(52) U.S. Cl. CPC A61M 1/127; A61M 1/1086; A61M 1/122; H04W 76/10; H04L 12/189; H04W 76/30; A61M 2205/3368; H02J 50/10; A61M 2205/3523; A61M 2205/3592; A61M 2205/8243; A61M 2205/52; A61M 2205/3365; H02J 50/80

(57) ABSTRACT

An internal controller implantable within the body of a patient as part of a left ventricular assist device (LVAD) system and a method therefore are provided. According to one aspect, the internal controller includes processing circuitry configured to establish a radio frequency (RF) communication session with a first external power transmitter that responds to the advertisement. The processing circuitry is also configured to determine when a power transmission status of the first external power transmitter does not match a power receipt status of the internal controller, and then terminate the RF communication session with the first external power transmitter and cause the radio interface to broadcast another advertisement.

### CROSS-REFERENCE TO RELATED APPLICATION

n/a

### FIELD

The present technology is generally related to implantable medical devices such as a left ventricular assist device (LVAD), and more particularly to an internal controller configured to select between two or more external power transmitters and an external power transmitter configured to respond to an internal controller so configured.

### BACKGROUND

Referring to FIG. 1, an implantable LVAD system 10 has internal components (in the body of the patient) and external components. The LVAD system 10 may typically include an LVAD pump 12 an implanted controller (i-controller) 14 having an internal battery 16, an implanted internal transcutaneous energy transfer system (TETS) coil (i-coil) 18, an external TETS coil (e-coil) 20 and an external power transmitter 21 with a detachable battery 24. In operation, power is supplied from the external power transmitter 21 to the i-controller 14 via mutual coupling of the coils 18 and 20, in order to charge the internal battery 16 of the i-controller 14 and to power the LVAD pump 12. The coils 18 and 20 transfer power by mutual induction of electromagnetic energy over the air and through the body. The power supplied by the external power transmitter 21 may come from the detachable battery 24 or from a wall outlet, for example.

### SUMMARY

The techniques of this disclosure generally relate to an internal controller configured to select between two or more external power transmitters and an external power transmitter configured to respond to an internal controller so configured.

According to one aspect, an internal controller of an implanted medical device includes an internal radio interface and processing circuitry. The internal radio interface is configured to broadcast an advertisement. The processing circuitry is configured to establish a radio frequency (RF) communication session with a first external power transmitter that responds to the advertisement. The processing circuitry is further configured to determine if the first external power transmitter is providing power to the internal controller. When a power transmission status of the first external power transmitter does not match a power receipt status of the internal controller, then the processing circuitry is configured to terminate the RF communication session with the first external power transmitter and cause the radio interface to broadcast another advertisement.

According to this aspect, in some embodiments, when the processing circuitry and internal radio interface are configured to engage in the RF communication session, the processing circuitry is further configured to detect an onset of receiving power from any external power transmitter; and in response to detecting the onset of receiving power from any external power transmitter, terminate the RF communication session with the first external power transmitter. In some embodiments, when there is a loss in RF connection between the first external power transmitter and the internal controller, the processing circuitry is further configured to terminate the RF communication session. In some embodiments, when the implanted medical device is receiving power but the first external power transmitter indicates by RF communication that the first external power transmitter is not transmitting power, then the processing circuitry is further configured to: terminate the RF communication session with the first external power transmitter; and commence transmitting advertisements. In some embodiments, when the implanted medical device is not receiving power and when the first external power transmitter indicates by RF communication that the first external power transmitter is not transmitting power and when the implanted medical device is engaged in an RF communication session with the first external power transmitter, then the processing circuitry is configured to maintain the RF communication session. In some embodiments, the processing circuitry is configured to engage in an RF communication session with an external power transmitter that first responds to the advertisement with an indication of a readiness to transmit power to the internal controller. In some embodiments, when the internal controller is in an RF communication with the first external power transmitter and when power is provided by a second external transmitter, then the processing circuitry is configured to terminate the RF communication session with the first external power transmitter. In some embodiments, when neither the first external power transmitter and a second external power transmitter are providing power to the internal controller and when the internal controller is in an RF communication session with the first external power transmitter, then the processing circuitry is configured to maintain the RF communication session with the first external power transmitter.

According to another aspect, a method in an internal controller of an implanted medical device includes broadcasting an advertisement. The method further includes establishing a radio frequency (RF) communication session with a first external power transmitter that responds to the advertisement. The method also includes determining if the first external power transmitter is providing power to the internal controller. The method also includes, when a power transmission status of the first external power transmitter does not match a power receipt status of the internal controller, then terminating the RF communication session with the first external power transmitter and causing the internal radio interface to broadcast another advertisement, unless power transmission is lost, in which case the RF communication session is not terminated. In some embodiments, when the internal controller is configured to engage in the RF communication session, then, the method includes detecting an onset of receiving power from any external power transmitter; and in response to detecting the onset of receiving power from any external power transmitter, terminating the RF communication session with the first external power transmitter.

According to this aspect, in some embodiments, when there is a loss in RF connection between the first external power transmitter and the internal controller, then terminating the RF communication session. In some embodiments, when the implanted medical device is receiving power but the first external power transmitter indicates by RF communication that the first external power transmitter is not transmitting power, then: terminating the RF communication session with the first external power transmitter; and commencing transmitting advertisements. In some embodiments, when the implanted medical device is not receiving power and when the first external power transmitter indicates by RF communication that the first external power transmitter is not transmitting power and when the implanted medical device is engaged in an RF communication session with the first external power transmitter, then maintaining the RF communication session. In some embodiments, the method further includes engaging in an RF communication session with an external power transmitter that first responds to the advertisement with an indication of a readiness to transmit power to the internal controller. In some embodiments, when the internal controller is in an RF communication with the first external power transmitter and when power is provided by a second external transmitter, then terminating the RF communication session with the first external power transmitter. In some embodiments, when neither the first external power transmitter and a second external power transmitter are providing power to the internal controller and when the internal controller is in an RF communication session with the first external power transmitter, then maintaining the RF communication session with the first external power transmitter.

According to yet another aspect, an external power transmitter in communication with an internal controller of an implanted medical device is provided. The external power transmitter includes processing circuitry configured to: receive an advertisement from the internal controller; respond to the advertisement when a power transmission status of the external power transmitter matches a power receipt status of the internal controller and delay responding to the advertisement when the power transmission status of the external power transmitter does not match the power receipt status of the internal controller; and establish a radio frequency (RF) communication session between the internal controller and the external power transmitter.

According to this aspect, in some embodiments, the communication session is terminated in response to a signal from the internal controller when the internal controller is receiving power from another external power transmitter.

According to another aspect, a method in an external power transmitter in communication with an internal controller of an implanted medical device is provided. The method includes receiving an advertisement from the internal controller, responding to the advertisement upon receipt of the advertisement when a power transmission status of the external power transmitter matches a power receipt status of the internal controller and delaying responding to the advertisement when the power transmission status of the external power transmitter does not match the power receipt status of the internal controller, and establishing a radio frequency (RF) communication session between the internal controller and the external power transmitter.

According to this aspect, in some embodiments, the communication session is terminated in response to a signal from the internal controller when the internal controller is receiving power from another external power transmitter.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a block diagram of an implanted medical device system;

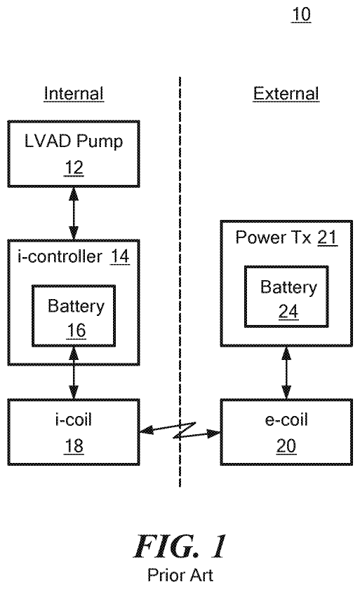

FIG. 2 is a block diagram of an embodiment of an implanted medical device system that implements a process of selecting one of a plurality of external power transmitters;

FIG. 3 is a block diagram of an implanted medical device system that includes a mobile device according to principles set forth herein;

FIG. 4 is a flowchart of a process in an external power transmitter for receiving an advertisement and conditionally delaying a response to the advertisement according to principles set forth herein;

FIG. 5 is a flowchart of a first process implemented by an internal controller according to principles set forth herein;

FIG. 6 is a flowchart of a second process implemented by an internal controller according to principles set forth herein;

FIG. 7 is a flowchart of a third process implemented by an internal controller according to principles set forth herein;

FIG. 8 is a block diagram of an internal controller and two external power transmitters exchanging messages and setting up a radio frequency (RF) communication session according to principles set forth herein;

FIG. 9 is a state diagram of states of an internal controller according to principles set forth herein; and

FIG. 10 is a flow chart of an exemplary process in an internal controller according to principles set forth herein.

### DETAILED DESCRIPTION

Patients having an implanted medical device such as an LVAD may sometimes have more than one external power transmitters to supply power to, and control, the implanted medical device. In the case of two external power transmitters, one power transmitter is the “primary” that is very commonly used and one is the “backup” that is relatively rarely used. When a transition between primary and backup occurs, the system needs to manage the RF connection to ensure that data and system status are appropriately displayed on the power transmitter that is in active use.

The power transmitters may be used interchangeably at the discretion of the patient. For example, the two power transmitters may be positioned for convenience in two different locations of the patient's home. In this case, the two power transmitters may both be used frequently. If the transition between the use of two transmitters is not coordinated, significant confusion of the patient may result, because the display on the power transmitter providing power would show no information because the RF session is with the other power transmitter which may not be in view of the patient.

Some embodiments described herein are related to an internal controller configured to select between two or more external power transmitters and an external power transmitter configured to respond to an internal controller so configured. In some embodiments, methods are provided to enable the internal controller to select an external power transmitter among a plurality of external power transmitters so as to avoid a condition of being in an RF communication session with one external power transmitter while receiving power from a second external power transmitter.

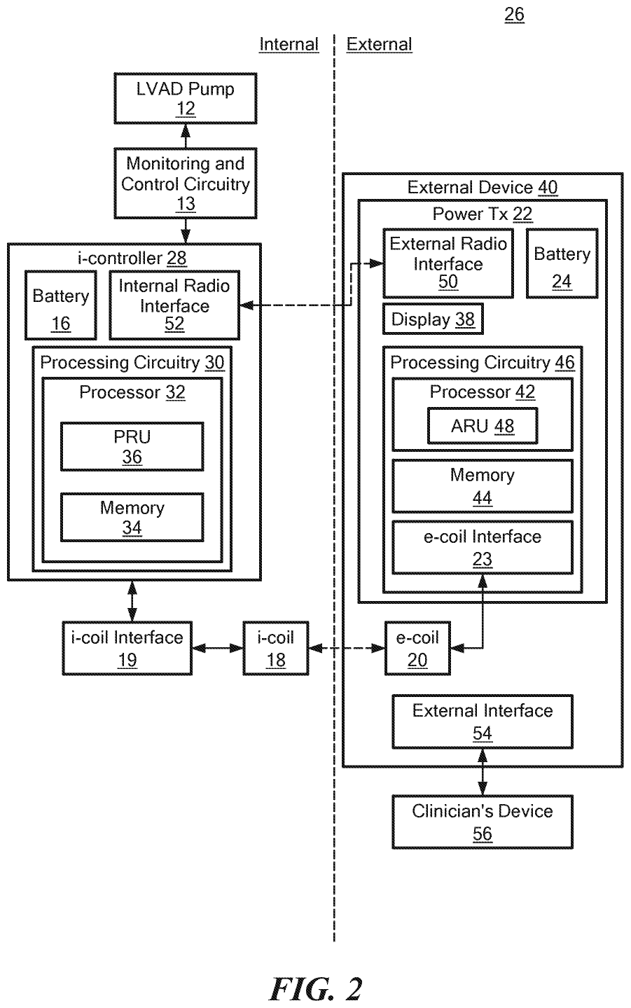

FIG. 2 shows a block diagram of one example configuration of an implanted medical device system 26 having external components such as an external power transmitter 22, and internal components such as an internal controller (i-controller) 28 configured to perform functions described herein. As used herein, the term “implanted medical device system 26” refers to the system that includes both the implanted/implantable components as well as external components described herein.

The i-controller 28 may have processing circuitry 30 which may include a processor 32 and an internal memory 34 and/or controller firmware. The processor 32 may be configured to execute computer instructions stored in the internal memory 34. Those instructions may include instructions to cause the processor to perform some of the processes described in more detail below. The processor 32 may therefore implement a power transmitter rejection unit (PRU) 36 configured to select an external power transmitter 22 with which to establish a radio frequency (RF) communication session.

A message or result from the i-controller 28 may be transferred from the i-controller 28 to an external display 38 of an external device 40, which may include a processor 42 and a memory 44 within processing circuitry 46, the external power transmitter 22 and the detachable battery 24, as well as the e-coil 20 in some embodiments. The memory 44 may be configured to store computer instructions to be executed by the processor 42 and data for processing according to principles set forth herein. The processor 42 may implement an advertisement response unit (ARU) 48 configured to respond to an advertisement from the i-controller 28 immediately when providing power and to delay its response if it is not providing power to the i-controller 28. The external display 38 may be configured to display information received from the i-controller 28.

Electrical communication of signals and power between the internal components of i-controller 28 may be via communication busses and individual electrical conductors not shown in FIG. 2. For example, a multi-conductor address bus and data bus may connect processor 32 with internal memory 34. In some embodiments, an i-coil interface 19 associated with i-coil 18 may be included in the set of internal components making up the implanted medical device system 26. One purpose of i-coil interface 19 may be to modulate the alternating current applied to the i-coil 18 with signals from the i-controller 28 to be transmitted from the i-coil 18 to the e-coil 20 and/or to demodulate signals to be received by the i-coil 18 from the e-coil 20. In some embodiments, a purpose of the i-coil interface 19 is to provide conversion between the alternating current (AC) of the i-coil 18 and direct current (DC) to charge the battery 16.

The power supplied to the i-coil 18 may be adjusted by varying the AC electrical current in the e-coil 20. Some or all functions of the i-coil interface 19 may be included in the i-controller 28 and/or the i-coil 18. In some embodiments, the i-coil 18 and/or i-coil interface 19 may be internal to or considered part of the internal controller 28. Similarly, electrical communication of signals and power between the internal components of external device may be by communication busses and individual electrical conductors not shown in FIG. 2. For example, a multi-conductor address bus and data bus may connect processor 42 with memory 44. In some embodiments, an e-coil interface 23 associated with e-coil 20 may be included in the set of external components making up the implanted medical device system 26. The e-coil interface 23 may include a TETS interface configured to demodulate information signals from the processing circuitry 30 transmitted from the i-coil 18 to the e-coil 20. The e-coil interface 23 may also be configured to couple power from the external power transmitter 22 to the e-coil 20. In some embodiments, the e-coil interface 23 may be two distinct units, one unit for demodulation of signals from the i-controller that are uploaded via the coils 18 and 20, and one unit for coupling power from the external power transmitter 22 to the e-coil 20. In some embodiments, the i-controller 28 may upload information to the external power transmitter 22 via the coils 18 and 20, but the power transmitter does not download information to the i-controller 28 via the coils 18 and 20.

In some embodiments, the internal components of the implanted medical device system 26 may include monitoring and control circuitry 13. A purpose of monitoring and control circuitry 13 may include monitoring speed and temperature, for example, of the LVAD pump 12. Another purpose of the monitoring and control circuitry 13 may include controlling the speed of the LVAD pump 12. In some embodiments, some or all of the monitoring and control circuitry 13 may be incorporated into the LVAD pump 12 and/or the i-controller 28. In some embodiments, some or all of the functions performed by the monitoring and control circuitry 13 may be performed by the processing circuitry 30. Thus, in some embodiments, the monitoring and control circuitry 13 may include one or more temperature sensors embedded in the LVAD pump 12 or the i-controller 28. Information obtained from and/or about the LVAD pump 12, such as speed and temperature, may be sent to the external device 40 to be displayed by external display 38.

The various internal components making up the LVAD system may be grouped into one or more separate housings. Similarly, the various external components making up the LVAD system may be grouped into one or more separate housings. Further, some of the components shown and described as being internal to the i-controller 28 may be instead, external to i-controller 28 in some embodiments. Similarly, some of the components shown and described as being internal to the external device 40 may be instead, external to external device 40, in some embodiments. Note further that some of the functions performed by processor 32 may be performed instead by processor 42.

Note that transfer of information from the external device 40 to the internal memory 34, and vice versa, may be by wireless radio frequency (RF) transmission (over the air and through the body when the i-controller 28 is implanted). Accordingly, in some embodiments, the external device 40 includes an external radio interface 50 and the i-controller 28 includes an internal radio interface 52. In some embodiments, the external radio interface 50 and the internal radio interface 52 are RF transceivers having both an RF receiver for receiving information wirelessly and an RF transmitter for transmitting information wirelessly. Such RF transceivers may be Bluetooth and/or Wi-Fi compliant, for example. In some embodiments, the RF receiver and RF transmitter within the external device 40 or within the i-controller 28 are integrated into one unit, whereas in some embodiments, they could be physically separate units.

Also, information may be communicated to the i-controller 28 from the external power transmitter 22 via the coils 18 and 20, by modulating a parameter of power transmission, such as modulating the frequency of the transmitted power, or by modulating a parameter of the i-coil interface 19, for example, by modulating a tuning capacitance of the i-coil interface 19 or by modulating the load level of the i-controller and/or the i-coil interface 19.

The external device 40 could be a patient's external device that has an external interface 54 which provides an interface between the external device 40 and a clinician's device 56. The clinician's device might, for example, have a USB port and interface 54 might include a USB port, so that a USB cable may connect the two ports. The clinician's device 56 may read data from the external device 40 and write information and control signaling to the external device 40, in some embodiments. In the alternative to a wireline connection, the interface 54 could include or be a radio interface.

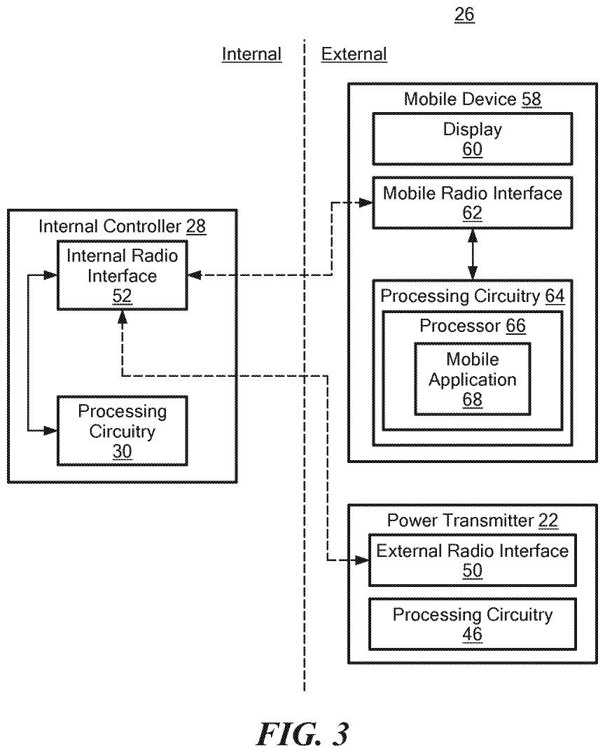

FIG. 3 is a block diagram of an implanted medical device system 26 that includes a mobile device 58 with a mobile application 68 in wireless communication with the i-controller 28. The mobile device 58 may be a mobile phone or other mobile digital device that can process information and communicate wirelessly with the i-controller. Accordingly, the mobile device 58 has a display 60, a mobile radio interface 62, processing circuitry 64, processor 66 which runs the mobile application 68. The radio interfaces 50, 52 and 62 may be Bluetooth Low Energy compatible radio interfaces, and the i-controller 28 may be a peripheral device responsible for advertising, while the mobile device 58 and the external power transmitter 22 may operate as master or central devices responsible for scanning and issuing connection requests.

Communication from the i-controller 28 to the external power transmitter 22 enables the display on external display 38 of implanted device information such as pump data and alarm indications. The i-controller 28 may exchange, via the radio interfaces 50 and 52, diagnostic and log file data with the external power transmitter 22. The i-controller 28 may receive programming commands from an external device such as the clinician's device 56 or mobile device 58. Further, communication from the i-controller 28 to the mobile device 58, via the radio interfaces 52 and 62, enables remote monitoring in cases where the mobile device 58 is connected to the Internet, and enables the display 60 to display information about the state of the implanted portion of the implanted medical device system 26 such as, for example, remaining battery runtime. In some embodiments, the internal radio interface 52 may only communicate with the external radio interface 50 and the mobile radio interface 62 one at a time. In some embodiments, when the i-controller 28 is not engaged in a communication session with an external device, such as external power transmitter 22 or mobile device 58, the i-controller 28 may advertise continually to enable rapid reestablishment of the wireless connection between the i-controller 28 and the external power transmitter 22 or mobile device 58. Conversely, either one or both of the external power transmitter 22 or mobile device 58 may scan for such advertisements.

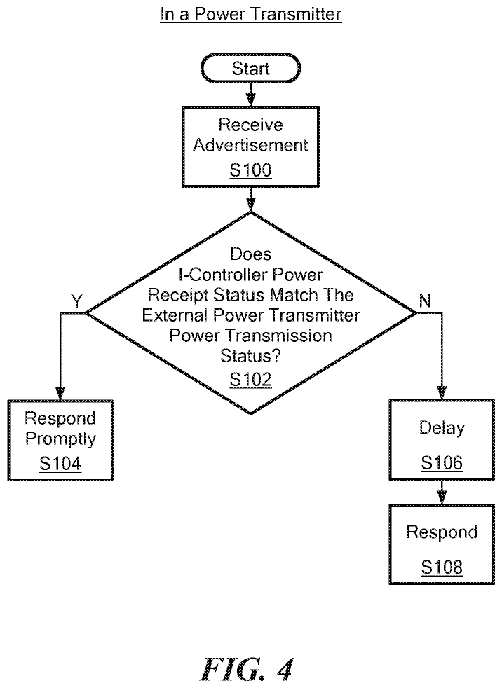

FIG. 4 is a flowchart of an exemplary process implemented in an external power transmitter 22 according to principles set forth herein. The process begins with the external power transmitter 22 receiving, via the external radio interface 50, an advertisement sent from an i-controller 28 via the internal radio interface 52 (Block S100). The advertising response unit (ARU) 48 of the external power transmitter 22 determines if the external power transmitter 22 power transmission status (whether the external power transmitter 22 is providing TETS power) matches the i-controller 28 power receipt status (whether the i-controller 28 is receiving power). If so, the external power transmitter 22 promptly sends to the i-controller 28 a response to the advertisement (Block S104). If the transmission and receipt statuses do not match, then the external power transmitter 22 delays (Block S106) before sending the response to the advertisement (Block S108). The power receipt status of the i-controller 28 is transmitted in the advertisement. As used herein, the power transmission status of the external power transmitter 22 is said to match the power receipt status of the i-controller 28 when the external power transmitter 22 is providing power and the i-controller 28 is receiving power or when the external power transmitter 22 is not providing power and the i-controller 28 is not receiving power. The power transmission status is said to not match the power receipt status when the when the external power transmitter 22 is providing power and the i-controller 28 is not receiving power or when the external power transmitter 22 is not providing power and the i-controller 28 is receiving power.

As long as the i-controller 28 is not in an RF communication session with an external power transmitter 22, the i-controller will establish an RF communication session based on a valid response from the Power Transmitter at any time. The time delay is the mechanism that prioritizes communication connection with the external power transmitter 22 that is providing power. If neither external power transmitter 22 is providing power, then neither external power transmitter 22 will delay its response and the connection is established on a first come first served basis—there is no priority control mechanism in this case, in some embodiments.

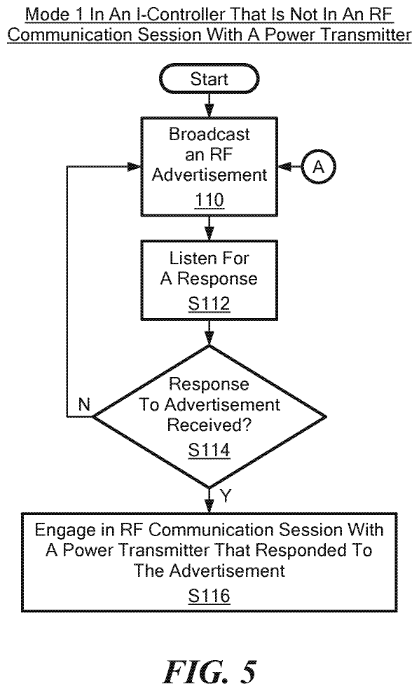

FIG. 5 is a flowchart of a process implemented by a PRU 36 of an i-controller 28 of an implanted medical device operating in a first mode when the i-controller 28 is not in an RF communication session with a power transmitter. The i-controller 28 broadcasts an advertisement on the RF channel via the internal radio interface 52 (Block S110). The i-controller 28 then listens for a response (Block S112). If a response to the advertisement is received (Block S114), then the i-controller 28 engages in an RF communication session with a power transmitter that responded to the advertisement (Block S116).

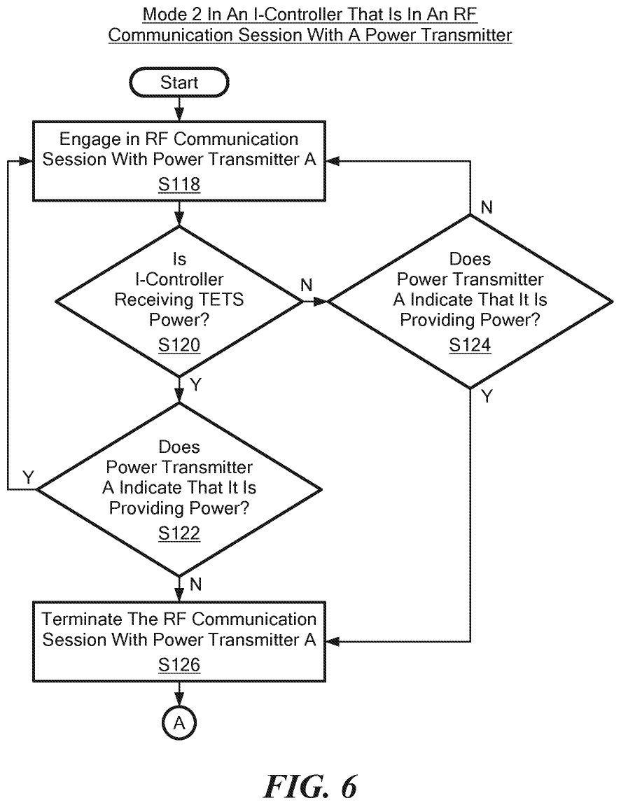

FIG. 6 is a flowchart of a process implemented by a PRU 36 in an i-controller 28 of an implanted medical device operating in a second mode when the i-controller 28 is in an RF communication session with an external power transmitter 22. The i-controller 28 engages in RF communication with a first external power transmitter 22 A (Block S118). If the i-controller 28 is receiving TETS power (Block S120) and a first external power transmitter 22A indicates that it is providing power (Block S122), then the i-controller continues to engage in the RF communication session with the first external power transmitter 22A (Block S118). If the i-controller 28 is not receiving TETS power (Block S120), and the first external power transmitter 22A indicates that it is providing power (Block S124), then the i-controller 28 terminates the RF communication session with the first external power transmitter 22A (Block S126 and returns to Block S110 of FIG. 5 to broadcast another advertisement. When the i-controller 28 is receiving TETS power (Block S120) and the first external power transmitter 22A indicates that it is providing power (Block S122), then the i-controller 28 continues to engage in the RF communication session with the external power transmitter 22A (Block S118).

Some embodiments address the following scenario:
A first i-controller A is in an RF communication session with a first external power transmitter A that is not providing power;
A second i-controller B is in an RF communication session with a second external power transmitter B that is not providing power;
For some reason the transmitting coils get placed in proximity to the incorrect devices resulting in:
   The first i-controller A is now receiving power from power transmitter B
   The second i-controller B is now receiving power from power transmitter A
      By designing the external power transmitter 22 and i-controller 28 so that the i-controller 28 returns to advertising at a new onset of power delivery this situation is avoided.

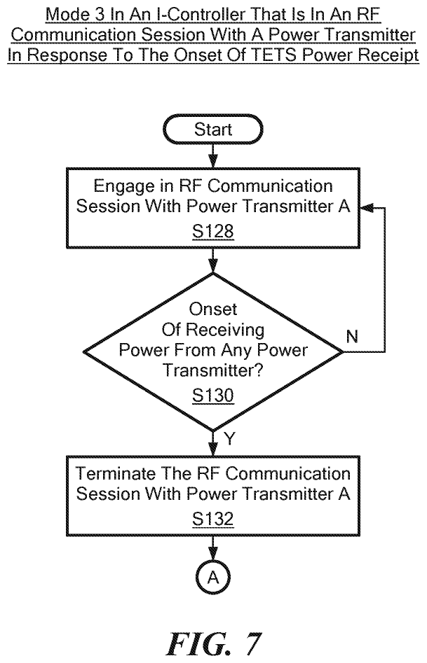

FIG. 7 is a flowchart of a process implemented by the PRU 36 in an i-controller 28 of an implanted medical device operating in a third mode when the i-controller 28 is in an RF communication session with an external power transmitter 22 and responds to the onset of TETS power receipt via the i-coil 18. The i-controller 28 engages in an RF communication session with a first external power transmitter 22A (Block S128). At the onset of receiving TETS power from any external power transmitter 22 (Block S130), the i-controller 28 terminates the RF communication session with the first external power transmitter 22A (Block S132). The i-controller 28 then returns to Block S110 of FIG. 5 to broadcast another advertisement.

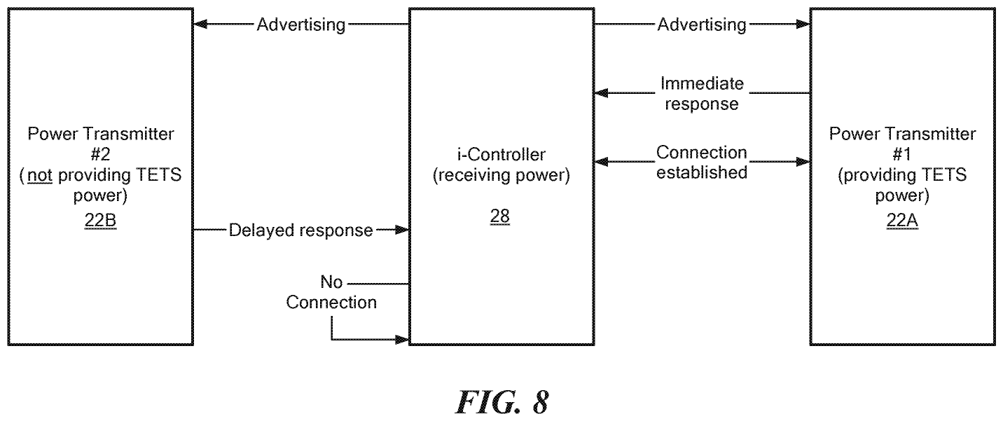

FIG. 8 is a block diagram illustrating a process of establishing an RF communication session between an i-controller 28 and an external power transmitter 22 when the i-controller 28 is receiving power. At the top of FIG. 8 and moving downward, which corresponds to increasing time, the process begins with the i-controller 28 broadcasting an advertisement to a first external power transmitter 22A and a second external power transmitter 22B. In the example, of FIG. 8, the first external power transmitter 22A is providing TETS power and the second external power transmitter 22B is not providing TETS power. Thus, the first external power transmitter 22A sends an immediate response to the advertisement and the second external power transmitter 22B does not send an immediate response to the advertisement. Consequently, an RF communication session is established between the i-controller 28 and the first external power transmitter 22A and an RF communication session is not established between the i-controller 28 and the second external power transmitter 22B.

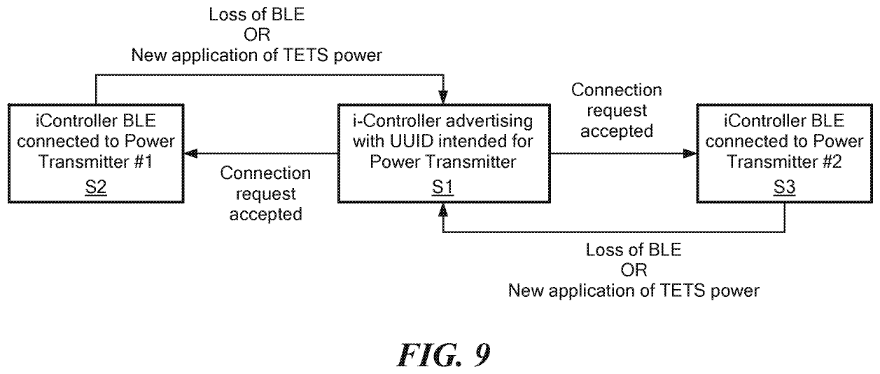

FIG. 9 is a state diagram showing different states of the i-controller 28. In State 51, the i-controller 28 advertises to any power transmitter. If a connection request from a first external power transmitter 22A (transmitter #1) is accepted by the i-controller 28, the i-controller 28 transitions to connection State S2 and an RF communication session is established between the i-controller 28 and the first external power transmitter 22A. If the first external power transmitter 22A is providing a new application of TETS power, or if the RF connection between the external radio interface 50 and the internal radio interface 52 is lost, the i-controller 28 transitions back to State 51 Similarly, if a connection request from a second external power transmitter 22B (transmitter #2) is accepted by the i-controller 28, the i-controller 28 transitions to connection State S3 and an RF communication session is established between the i-controller 28 and the second external power transmitter 22B. If the second external power transmitter 22B is providing a new application of TETS power, or if the RF connection between the external radio interface 50 and the internal radio interface 52 is lost, the i-controller 28 transitions back to State 51.

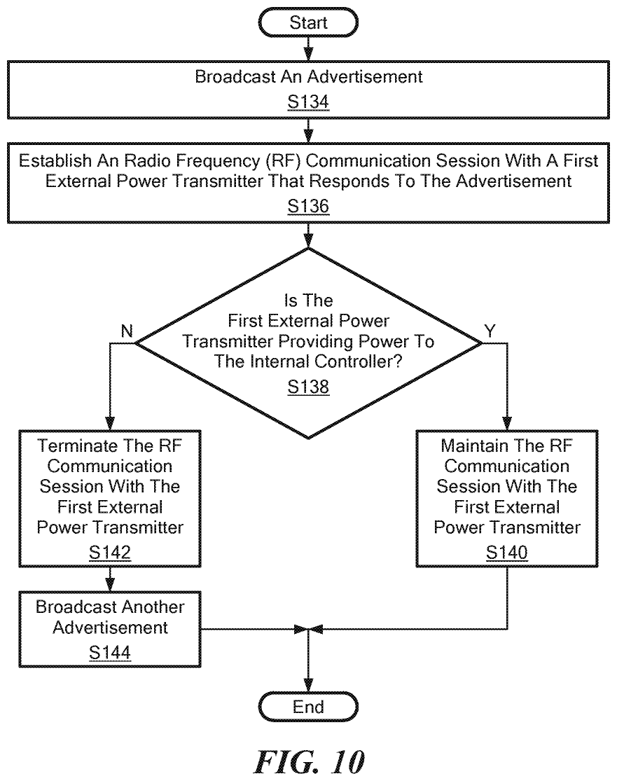

FIG. 10 is a flowchart of an example process in an i-controller 28 according to principles set forth herein when the i-controller 28 is receiving TETS power from an external power transmitter 22. An advertisement is broadcast (Block S134). An RF communication session is established with a first external power transmitter 22 that responds to the advertisement (Block S136). A determination is made whether the first external power transmitter 22 is providing power to the i-controller 28 (Block S138). If the first external power transmitter 22 is providing power, then the RF communication session is maintained (Block S140). Otherwise, the RF communication session is terminated (Block S142) and another advertisement is broadcast (Block S144).

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media and memory may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. An internal controller of an implanted medical device, the internal controller comprising:
an internal radio interface configured to broadcast an advertisement;
processing circuitry configured to:
establish a radio frequency (RF) communication session with a first external power transmitter that responds to the advertisement;
determine if the first external power transmitter is providing power to the internal controller; and
when a power transmission status of the first external power transmitter does not match a power receipt status of the internal controller, then terminate the RF communication session with the first external power transmitter and cause the radio interface to broadcast another advertisement.

2. The internal controller of claim 1, wherein, when the processing circuitry and internal radio interface are configured to engage in the RF communication session, the processing circuitry is further configured to:
detect an onset of receiving power from any external power transmitter; and
in response to detecting the onset of receiving power from any external power transmitter, terminate the RF communication session with the first external power transmitter.

3. The internal controller of claim 1, wherein, when there is a loss in RF connection between the first external power transmitter and the internal controller, the processing circuitry is further configured to terminate the RF communication session.

4. The internal controller of claim 1, wherein, when the implanted medical device is receiving power but the first external power transmitter indicates by RF communication that the first external power transmitter is not transmitting power, then the processing circuitry is further configured to:
terminate the RF communication session with the first external power transmitter; and
commence transmitting advertisements.

5. The internal controller of claim 1, wherein, when the implanted medical device is not receiving power and when the first external power transmitter indicates by RF communication that the first external power transmitter is not transmitting power and when the implanted medical device is engaged in an RF communication session with the first external power transmitter, then the processing circuitry is configured to maintain the RF communication session.

6. The internal controller of claim 1, wherein the processing circuitry is configured to engage in an RF communication session with an external power transmitter that first responds to the advertisement with an indication of a readiness to transmit power to the internal controller.

7. The internal controller of claim 1, wherein, when the internal controller is in an RF communication with the first external power transmitter and when power is provided by a second external transmitter, then the processing circuitry is configured to terminate the RF communication session with the first external power transmitter.

8. The internal controller of claim 1, wherein, when neither the first external power transmitter and a second external power transmitter are providing power to the internal controller and when the internal controller is in an RF communication session with the first external power transmitter, then the processing circuitry is configured to maintain the RF communication session with the first external power transmitter.

9. A method in an internal controller of an implanted medical device, the method comprising:
broadcasting an advertisement;
establishing a radio frequency (RF) communication session with a first external power transmitter that responds to the advertisement;
determining if the first external power transmitter is providing power to the internal controller; and
when the first external power transmitter is not providing power to the internal controller and when the internal controller is receiving power, then terminating the RF communication session with the first external power transmitter and causing the internal radio interface to broadcast another advertisement, unless power transmission is lost, in which case the RF communication session is not terminated.

10. The method of claim 9, wherein, when the internal controller is configured to engage in the RF communication session, then:
detecting an onset of receiving power from any external power transmitter; and
in response to detecting the onset of receiving power from any external power transmitter, terminating the RF communication session with the first external power transmitter.

11. The method of claim 9, wherein, when there is a loss in RF connection between the first external power transmitter and the internal controller, then terminating the RF communication session.

12. The method of claim 9, wherein, when the implanted medical device is receiving power but the first external power transmitter indicates by RF communication that the first external power transmitter is not transmitting power, then:
terminating the RF communication session with the first external power transmitter; and
commencing transmitting advertisements.

13. The method of claim 9, wherein, when the implanted medical device is not receiving power and when the first external power transmitter indicates by RF communication that the first external power transmitter is not transmitting power and when the implanted medical device is engaged in an RF communication session with the first external power transmitter, then maintaining the RF communication session.

14. The method of claim 9, further comprising engaging in an RF communication session with an external power transmitter that first responds to the advertisement with an indication of a readiness to transmit power to the internal controller.

15. The method of claim 9, wherein, when the internal controller is in an RF communication with the first external power transmitter and when power is provided by a second external transmitter, then terminating the RF communication session with the first external power transmitter.

16. The method of claim 9, wherein, when neither the first external power transmitter and a second external power transmitter are providing power to the internal controller and when the internal controller is in an RF communication session with the first external power transmitter, then maintaining the RF communication session with the first external power transmitter.

17. An external power transmitter in communication with an internal controller of an implanted medical device, the external power transmitter comprising processing circuitry configured to:
receive an advertisement from the internal controller;
respond to the advertisement upon receipt when a power transmission status of the external power transmitter matches a power receipt status of the internal controller, and delay responding to the advertisement when the power transmission status of the external power transmitter does not match the power receipt status of the internal controller; and
establish a radio frequency (RF) communication session between the internal controller and the external power transmitter.

18. The external power transmitter of claim 17, wherein the communication session is terminated in response to a signal from the internal controller when the internal controller is receiving power from another external power transmitter.

19. A method in an external power transmitter in communication with an internal controller of an implanted medical device, the method comprising:
receiving an advertisement from the internal controller;
responding to the advertisement upon receipt of the advertisement when a power transmission status of the external power transmitter matches a power receipt status of the internal controller and delaying responding to the advertisement when the power transmission status of the external power transmitter does not match the power receipt status of the internal controller; and
establishing a radio frequency (RF) communication session between the internal controller and the external power transmitter.

20. The method of claim 19, wherein the communication session is terminated in response to a signal from the internal controller when the internal controller is receiving power from another external power transmitter.
